(19) United States
(12) Patent Application Publication
(10) Pub. No.: US 2007/0003938 A1
(43) Pub. Date: Jan. 4, 2007

(54) Hybridization of genomic nucleic acid without complexity reduction

(75) Inventors: Glenn Fu, Dublin, CA (US); Heng Tao, Mountain View, CA (US)

Correspondence Address: BEYER WEAVER & THOMAS, LLP, P.O. BOX 70250, OAKLAND, CA, 94612-0250, US

(73) Assignee: Perlegen Sciences, Inc., Mountain View, CA (US)

(21) Appl. No.: 11/173,309

(22) Filed: Jun. 30, 2005

(51) Int. Cl. C12Q 1/68

(52) U.S. Cl. 435/6

(57) ABSTRACT

Disclosed are techniques for reliably detecting target sequences in a complex nucleic acid sample, typically in the range of about 400 MB or greater, without employing a complexity reduction technique. The method employs relatively high quantities of a hybridization competitor, e.g., multiple times the amount of nucleic acid sample present. When the sample and competitor come in contact with nucleic acid probes complementary to target sequences, for an appropriate length of time under defined hybridization conditions (buffer composition, temperature, etc.), the target and probe hybridize reliably.

### BACKGROUND

Methods, compositions, kits, and associated tools for hybridizing genomic nucleic acid samples are disclosed. In certain embodiments, whole genomes are hybridized without complexity reduction.

A challenge in modern genetic analysis involves reliably detecting (e.g., identifying and/or genotyping) individual SNPs (single nucleotide polymorphisms) and other features of a genome. The task may be analogized to finding a needle in a haystack, a very large haystack. Tools for genotyping individual organisms must detect many such SNPs or other pertinent genetic features efficiently and at low cost to have practical application. The entire genome of an organism is typically a starting point for such analysis. Because current analytic technologies are unable to return accurate results when tested against an entire genome, research to date has focused on modes of reducing “complexity” of the genome.

Complexity may be viewed as the amount or length of unique sequence in a genetic sample. A very long sample containing vast regions of simple repeat sequences has less complexity than a different, comparably long sample having few or no repeat sequences. The human genome has a complexity of approximately 3×10⁹ base pairs (3 GB).

A complex genome can be viewed as having regions or sequences of interest that can be detected as “target signal” relative to other regions or sequences that produce “background signal.” The target signal typically results from relatively short sequences that include the position of SNPs or other genetic loci to be assayed as well as sequences flanking them. The background signal is produced by the non-target content within the genome. Often, sequences giving rise to target signal are referred to as “target sequences.” The human genome presents a particularly complex sample for analysis. It appears to contain between about five million and about eight million Single Nucleotide Polymorphisms (SNPs) and its complexity is approximately 3×10⁹.

Typically, assaying involves contacting fragments of a sample with a microarray or other source of multiple short hybridization probes. Without complexity reduction, conventional assay techniques fail to reliably detect target sequences in highly complex samples. One of the main reasons why such techniques fail is non-specific binding of non-target sequences to probes; in a highly complex sample the overwhelming amount of “background signal” swamps the “target signal.”

As mentioned, effort to date in the field of high-complexity genetic analysis has focused on reducing the complexity of genomic samples. This is accomplished by increasing the ratio of target to non-target sequences, where the target sequences are those in a genomic sample that are to be analyzed and the non-target sequences are those in the genomic sample that are not to be analyzed. In general, the higher the ratio of target to non-target sequences, the more reliably the genomic sample can be assayed for the target sequences.

Unfortunately, complexity reduction comes at a cost. Conventionally, Polymerase Chain Reaction (PCR) is used to reduce complexity. PCR amplifies a pre-specified region or fragment of a nucleic acid sample. Over multiple cycles of denaturing and annealing, PCR generates many additional copies of a target fragment. In such cases, PCR effectively selects or isolates the pre-specified sequence of interest from the remainder of the nucleic acid sequence.

Often, in genotyping applications, PCR must amplify multiple distinct sequences within a nucleic acid sample. This becomes expensive and time consuming when there are a large number of sequences to amplify. Each sequence to be amplified requires its own unique set of PCR primers, which represents a significant cost in the process. Furthermore traditional PCR requires each sequence to be amplified in its own reaction vessel with its own PCR reactants, adding to the time and cost associated with PCR-based complexity reduction.

Multiplex PCR is a process that addresses some of the difficulties associated with traditional PCR. Multiplex PCR can amplify multiple sequences in a single reaction vessel. The vessel includes the sample under analysis, a unique primer set for each sequence to be amplified, as well as polymerase and deoxyribonucleotide triphosphates (dNTPs—e.g., dATP, dCTP, dGTP, and dTTP) that are shared by all amplification reactions. Thus, it has become possible to simultaneously amplify hundreds of sequences in a single reaction mixture. This can greatly improve efficiency.

However, multiplex PCR still requires a unique set of primers for each sequence to be amplified and therefore the cost of the procedure is nearly proportional to the number of sequences to be amplified or isolated. Further, in complex genomic analysis far more than a few hundred sequences must be amplified. To fully genotype an individual of a higher species requires amplification of many thousands or millions of sequences. Thus, many separate multiplex PCR reactions must be conducted. This process can still become very costly and time consuming even with the efficiency gains inherent in multiplex PCR.

Other complexity reduction techniques have comparable costs and inefficiencies. Complexity reduction techniques that are well known in the art include subtractive hybridization, size fractionation, (DOP)-PCR, denaturation/partial renaturation for removal of repeat sequences, the use of a Type IIs endonuclease combined with selective ligation, and arbitrarily primed PCR, some of which are detailed in, e.g., U.S. Pat. No. 6,361,947 and Jordan, et al. (2002) “Genome complexity reduction for SNP genotyping analysis”, Proc. Natl. Acad. Sci. U.S.A. 99(5):2942-7.

The inefficiencies and expense of traditional complexity reduction have led some researchers to seek alternative techniques. Such techniques may employ a hybridization “competitor” to reduce background hybridization of non-target sequences to hybridization probes. The competitor is a nucleic acid such as COT-1 DNA or herring sperm DNA, which hybridize to low complexity or repetitive sequences from a genomic nucleic acid sample and effectively reduce the amount of non-target sequences available for hybridizing with the probe. In other words, some of the sample fragments hybridize with the competitor and are temporarily unavailable for hybridizing with the probes. Of course, both target and non-target sequences of the sample can temporarily hybridize with the competitor, but the target sequences also have a hybridization partner (the probes) to which they can form relatively stable duplexes. This process effectively promotes the hybridization of target sequences to the correct probes.

The amount of competitor required for a given sample is related to the complexity of the sample. While hybridization competitors have been effective in some situations, those situations are limited to samples having a complexity of under approximately 400 MB, well below the complexity of the human genome. With more complex samples, researchers have attempted to use greater and greater quantities of competitor. However, at some point so much competitor is present that it interferes with hybridization of the target to complementary hybridization probes. Not only does it reduce background signal, but it also effectively reduces target signal. Further, high levels of competitor can also adversely affect the solubility, pH, and hybridization rate of the sample.

More effective nucleic acid analysis techniques that employ little or no complexity reduction would provide an important advance in the field.

### SUMMARY

The present invention provides methods, kits, compositions, apparatus, and the like for reliably detecting target sequences in a complex nucleic acid sample, typically in the range of 400 MB or greater, without employing a complexity reduction technique such as size fractionation, locus-specific PCR, subtractive hybridization, and the like. Methods employ relatively high quantities of a hybridization competitor, typically multiple times the amount of nucleic acid sample present. When the sample and competitor come in contact with nucleic acid probes complementary to target sequences, for an appropriate length of time under defined hybridization conditions (buffer composition, temperature, etc.), the target and probe hybridize reliably. The invention is particularly useful in analyzing large nucleic acid samples for SNPs or other features. For example, the invention may be employed to analyze nucleic acid samples with complexitites as high as 1 GB or even 3 GB and may be employed to analyze the whole human genome or a portion thereof.

In certain aspects of the invention, methods allow a complex genomic nucleic acid sample to reliably hybridize with one or more probes complementary to one or more target sequences within the genomic nucleic acid sample. One method of the invention may be characterized by the following sequence: (a) providing the genomic nucleic acid sample, wherein the genomic nucleic acid sample comprises a sequence with a complexity of at least about a 400 MB representing at least a portion of the genome of an organism; (b) contacting the genomic nucleic acid sample with a buffer solution comprising a competitor nucleic acid in an amount of at least about 30-fold greater than an amount (typically by mass) of the genomic nucleic acid sample; and (c) allowing the genomic nucleic acid sample in the buffer solution to contact the one or more probes and permit hybridization. In these methods, the genomic nucleic acid sample does not undergo complexity reduction before hybridization with the one or more probes.

The hybridization probes may be made available in many different forms and contexts. In some embodiments, the one or more probes are immobilized on at least one substrate such as one or more microarrays or collections of beads. Typically, the probes comprise multiple probes of distinct sequences immobilized on the one or more substrates. The probes may comprise oligonucleotides of between about 12 and 100 nucleotides in length, and in certain embodiments, between about 20 and 60 nucleotides in length.

As indicated, the hybridization competitor may be present in a relatively high concentration; e.g., between about 30-fold and 40-fold greater than the amount of the genomic nucleic acid sample. In certain embodiments, the concentration of competitor in the buffer solution is at least about 10 mg/ml. The competitor may have a relatively high solubility in the buffer solution, e.g., at least about 50 mg/ml. In certain embodiments, the competitor nucleic acid comprises RNA.

The buffer solution typically comprises one or more salts. In certain embodiments, the salt comprises a tetraalkylammonium salt such as TEACl (tetraethylammonium chloride) or TMACl (tetramethylammonium chloride). If TEACl is used, the buffer solution is typically maintained at a temperature of at most about 40 degrees C. during contact with the one or more probes. If TMACl is used, the buffer solution is maintained at a temperature of at most about 70 degrees C. during contact with the one or more probes. At these temperatures and buffer compositions, the genomic nucleic acid sample contacts the one or more probes for a time duration that is generally between about 10 and 100 hours.

Another aspect of the invention pertains to methods of preparing a complex genomic sample for analysis. Such methods may be characterized by the following operations: (a) providing a genomic nucleic acid sample having a complexity of at least about 4×10⁸ base pairs (at least about about 1×10⁹ base pairs in certain embodiments); (b) fragmenting the genomic nucleic acid sample to produce multiple fragments of the sample; (c) incorporating the fragments into a buffer solution; and (d) contacting the buffer solution with one or more hybridization probes and allowing the one or more hybridization probes to hybridize with the fragments of the genomic nucleic acid sample. The buffer solution comprises (i) a competitor nucleic acid serving as a hybridization competitor, and (ii) a salt which causes the fragments of the genomic nucleic acid to have a melting temperature of between about 20 and 70 degrees C. In certain embodiments, such as when there is insufficient sample at the beginning of the process, the method further comprises performing whole genome amplification on the genomic nucleic acid sample.

The sample may be fragmented by any appropriate method, including e.g., enzymatic or mechanical methods. In one embodiment, this involves contacting the genomic nucleic acid sample with a DNAse. In certain embodiments, the fragments of the sample have an average length of between about 50 and 500 base pairs. Note that in some embodiments, the nucleic acid sample is fragmented after it is incorporated in a buffer solution.

As indicated in the discussion of the previous method, the buffer solution salt may comprise a tetraalkylammonium salt such as tetraethylammonium chloride and/or tetramethylammonium chloride. In the former case, contacting the buffer solution with one or more hybridization probes may be carried out at a temperature of between about 20° C. and 40° C. In the latter case, contacting the buffer solution with one or more hybridization probes may be carried out at a temperature of between about 50° C. and 70° C. In certain embodiments, contacting the buffer solution with the one or more hybridization probes takes place for a period of between about 10 and 100 hours, often for a period of between about 20 and 70 hours.

Also as discussed above, the hybridization competitor typically has a high solubility (e.g., an RNA). In certain embodiments, it has a solubility of at least about 50 mg/ml of the buffer solution. In certain embodiments, the competitor is present in the buffer solution in a concentration of at least about 10 mg/ml, or at least about 30 mg/ml, or at least about 50 mg/ml, or at least about 70 mg/ml.

Depending on the type of hybridization probes employed and the context of the analysis, various techniques may be employed to detect hybridization. In certain embodiments, the sample nucleic acid is labeled, e.g., with a fluorescent or radioactive label to facilitate the detection of hybridization of the sample nucleic acid to one or more probes. In one embodiment, the method further comprises staining at least some fragments of the genomic nucleic acid samples which hybridized with the one or more hybridization probes.

A further aspect of the invention pertains to kits for analyzing a complex genomic nucleic acid sample. The kit may include the following components: (a) a hybridization competitor comprising a competitor nucleic acid; (b) a buffer salt comprising tetraethylammonium chloride; and (c) one or more probes complementary to one or more target sequences within the genomic nucleic acid sample. In certain embodiments, the kit may also include an enzyme for fragmenting the genomic nucleic acid sample such as a DNAse. In certain embodiments, the kit also includes a label (e.g., biotin) for fragments of the genomic nucleic acid sample. In certain embodiments, the kit may also include a stain for fragments of the genomic nucleic acid sample that hybridize with the one or more probes.

Other components of the kit may include one or more of the following: nucleic acid microarray, and instructions for preparing a buffer in which the competitor nucleic acid is present at a concentration of between about 10 mg/ml and about 100 mg/ml. In certain embodiments, instructions are provided for preparing a buffer in which the competitor nucleic acid is present at a concentration of at least about 30 mg/ml, or at least about 50 mg/ml, or at least about 70 mg/ml.

Yet another aspect of the invention pertains to a hybridization solution that may be characterized by following components: (a) a fluid medium; (b) a fragmented genomic nucleic acid sample of at least about 400 MB complexity in the liquid medium; (c) a hybridization competitor nucleic acid present in an amount of between about 30-fold and 40-fold greater than an amount of the genomic nucleic acid sample in the fluid medium; and (d) a buffer salt comprising tetraethylammonium chloride in the fluid medium.

These and other features and advantages of the present invention will be described in more detail below with reference to the associated drawings.

### BRIEF DESCRIPTION OF THE DRAWINGS

FIG. 1 is a process flow chart depicting a specific method for hybridizing a complex nucleic acid sample in accordance with an embodiment of this invention.

FIGS. 2A and 2B diagrammatically depict fragmentation of a nucleic acid strand into multiple fragments, some of which contain a target sequence of interest.

FIG. 3 is a flow chart depicting a whole genome amplification procedure that may be employed to increase the amount of genomic nucleic acid from a sample to be hybridized in accordance with embodiments of the invention.

FIG. 4 is a flow chart depicting an application of the present invention in which a single genomic sample and associated buffer solution is contacted with multiple different probe collections, each for different genetic markers. The buffer solution and sample need not be amplified or otherwise treated for complexity reduction before contact with any of the probe collections.

FIG. 5 presents a specific hybridization protocol in accordance with certain embodiments of the invention.

### DESCRIPTION OF CERTAIN EMBODIMENTS

Introduction and Overview

The disclosed methods, kits, compositions, apparatus, etc. involve hybridizing a nucleic acid sample in the presence of a competitor and under a defined set of hybridization conditions. The type and amount of competitor and the hybridization conditions are chosen to allow reliable hybridization between probes and target sequences within the sample. In certain embodiments, the competitor is present in an amount that is at least about 30-fold greater than the amount of nucleic acid sample, on a per unit mass basis. Despite such large quantities of competitor, the disclosed embodiments permit reliable hybridization of probe and target sequences. In certain embodiments, the hybridization conditions involve a defined minimum amount of time during which the buffer containing the sample contacts one or more hybridization probes. The length of time is a function of, at least, the probe(s), the sample, a buffer composition, and a hybridization temperature. In certain embodiments, the contact time at least about 10 hours, and sometimes at least about 25 hours.

One common application of hybridization is to utilize one or more probe sequences to detect one or more target sequences from a mixture of nucleic acid sequences that contains both target and non-target sequences. Certain embodiments described herein primarily concern hybridizing particular sequences contained within a whole genomic nucleic acid sample. As used herein, the term “whole genome nucleic acid” is understood to indicate all or substantially all of an organism's genomic DNA (or RNA), typically containing the loci for all SNPs or other features relevant to a particular assay. In specific embodiments, the disclosed techniques pertain to the whole genome of an organism or at least a portion thereof having a complexity of at least about 400 million base pairs (“400 megabases” or “400 MB”).

For convenience, the following description will sometimes refer to “DNA.” In such instances, it is intended that the description encompass any type of nucleic acid, whether naturally occurring, artificial or a combination thereof. And of course, RNA and cDNA are included within the scope of all such descriptions.

The process of hybridization is an interaction between two single-stranded nucleic acid strands to form a stable double-stranded nucleic acid. Of relevance to some of the techniques presented herein, hybridization may involve a single-stranded probe sequence and single-stranded target sequence. If either the probe or target sequences are originally double stranded, any one of a variety of techniques may be used to separate the double strands into single strands prior to hybridization. Many denaturization techniques are well-known in the field and may involve factors such as temperature, pH, etc.

A probe having a known or unknown nucleotide sequence is introduced, typically in a controlled manner, for assaying a sample. The sample comprises one or more nucleic acids (at least part of a genome in certain embodiments) comprising a large number of unknown or partially known nucleic acid sequences that may include both target and non-target sequences. In a typical assay, either the target or probe sequences are labeled for detection, generally by fluorescence or radioactivity.

In the hybridization reaction, target and probe sequences of nucleic acid that are complementary combine to form double strands of nucleic acid. Combinations of nucleic acids that can be formed by the hybridization reaction include, but are not limited to, DNA/DNA, DNA/RNA, RNA/RNA, and either DNA or RNA combined with or comprised of artificial (e.g., chemically synthesized, comprising nucleotide mimetics, etc.) oligonucleotides. These double strands can then be separated from the mixture of probe, target, and non-target genetic material and detected. The separation and detection process can be performed by any number of well-known techniques. One such technique is to bind the probe sequences to a substrate or fixed surface, such as the outer portion of a bead or a wafer, which is immersed in a sample comprising a mixture of target and non-target genetic material. Once the hybridization reaction takes place, the fixed surface is washed, retaining only target genetic material that has formed double strands with specific probes.

The target has a nucleic acid sequence that is complementary to the probe and, under the appropriate hybridization conditions, the probe and target will combine to form a double-stranded nucleic acid. One generally performs hybridization by introducing known probe sequences into a prepared sample that contains a mixture of both target and non-target sequences in order to determine the presence, concentration, and/or sequence of target sequences in the sample. As mentioned, genomic samples generally contain a relatively small amount of target sequence combined with a very large amount of non-target sequence. The invention is further applicable to situations where the ratio of target to non-target sequence is smaller than the range in which traditional hybridization techniques fail to discern the target sequence do to the overwhelming presence of non-target sequence in the sample. For example, typically traditional nucleic acid microarray hybridizations use sample of a complexity of about 40-50 MB, and sample with a complexity of greater than about 400 MB is not amenable to such analysis. However, the methods of the present invention allow analysis of sample with a complexity of about 3 GB on a nucleic acid microarray, an approximately ten-fold higher complexity than traditional methods typically allow.

As explained above, the complexity of a nucleic acid sample relates to the amount of unique sequence contained within the sample. As used herein, the term complexity sometimes refers to the ratio of target sequence to non-target sequence within a sample. Complexity reduction involves increasing the ratio of target to non-target sequences (or target to total sequences) in the sample. In other words, complexity reduction decreases the relative amount of unique sequence in a nucleic acid sample. Obviously, increasingly complex samples become increasingly more difficult to assay without significant complexity reduction. However, the methods presented herein do not require complexity reduction of nucleic acid samples prior to analysis via hybridization to probes, e.g., on a microarray.

As indicated, a fundamental problem with complex samples is that the non-target portions of a sample can swamp the probe hybridization process by non-specific annealing. Individual probes hybridize most strongly with perfectly complementary target sequences. While non-target sequences will not hybridize as strongly, the ratio of target to non-target sequences may be so small in highly complex samples that they greatly reduce the likelihood that a target sequence will be bound to a probe at any given instant in time. The problem may be viewed in terms of the relative rates of annealing non-target and target sequences to a probe. The rate is a strong function of the concentration of the annealing species, and because the concentration of non-target sequences is so much greater than the concentration of target sequences, it is not surprising that the non-target sequences can dominate the process. This can be understood intuitively by considering that there are many non-target sequences readily available to hybridize with the probe, even if only weakly. If a weakly bound non-target sequence peels off the probe, it will most likely be replaced by another non-target sequence in close proximity to the target. And even if some target sequences do hybridize with a complementary probe, they will not reside there forever and the equilibrium concentration of hybridized target sequences will remain relatively low, even after a very long annealing time. As a result of all this, the background due to non-specific binding is very high in highly complex samples.

Hybridization Conditions

A hybridization buffer creates the chemical conditions needed for hybridization to occur. In this invention, the buffer is intended to facilitate hybridization in highly complex samples using large quantities of competitor. The buffer may also be designed to provide a relatively low hybridization temperature.

The hybridization conditions are typically stringent. Hybridizing of a target sequence to a probe nucleotide sequence under stringent conditions occurs only when the target sequence is complementary to the probe nucleotide sequence. Stringent conditions are conditions under which a probe specifically hybridizes to a complementary target sequence, but only weakly to other sequences. Stringent conditions are sequence-dependent and vary by circumstance. Generally, stringent conditions are selected to be a few degrees lower (e.g., about 5° C.) than the thermal melting point (Tm) for the specific sequence at a defined ionic strength and pH. The Tm is the temperature (under defined ionic strength, pH, and nucleic acid concentration) at which 50% of the probes complementary to the target sequence anneal to the target sequence at equilibrium. (As the target sequences may be present in excess, at Tm, 50% of the probes are theoretically occupied at equilibrium.) Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations.

Generally, any buffer salt employed in conventional hybridization assays can be employed with this invention. However, specific embodiments of the invention employ specific buffer salts optimized for hybridization times that are significantly longer than those used by conventional assay techniques. The use of long hybridization times can create problems with the thermal breakdown of the target sequence, the probes, and the substrate used for hybridization. In order to overcome these problems, specific embodiments of the invention use a low-temperature hybridization buffer that permits hybridization to occur at a temperature below about 40° C., or below about 30° C. Such buffers employ a buffer salt that produces a relatively low Tm for the nucleic acid under investigation. Suitable buffer salts include tetraalkylammonium halides such as TEAC1. The final concentration of such buffer salts in the buffer and sample solution is typically between about 1 and 5M, and often between about 2 and 3M. In one embodiment, the hybridization buffer is comprised of a final concentration of 2.4M tetraethylammonium chloride (TEAC1), 0.05M tris hydrochloride, 0.05 nM control oligonucleotides, and 0.01% TritonX-100. In another embodiment, a buffer permits hybridization to occur below about 50° C. and employs a TMACl buffer salt. In a specific example, the hybridization buffer is comprised of a 3M final concentration of tetramethylammonium chloride (TMACl), 0.05M tris hydrochloride, 0.05 nM control oligonucleotides, and 0.01% TritonX-100.

As indicated, the hybridization solutions of this invention employ hybridization competitors, which non-specifically bind to fragments of the DNA sample. The competitor can be any natural or artificially produced RNA, DNA, or collection of synthetic nucleotides. A non-exclusive list of competitors includes Cot1 DNA, herring sperm DNA, human DNA, calf DNA, bacterial DNA, yeast RNA, salmon sperm DNA, poly-deoxyribonucleotides, and ribonucleotides. In some embodiments of the invention a large amount of competitor is required, which may necessitate the use of a highly soluble competitor. RNA based competitors are often used in these embodiments because RNA is significantly more soluble in aqueous media than DNA. In certain embodiments, the ratio of competitor to sample genomic DNA is between about 20:1 and 100:1 on a weight/weight basis, with certain embodiments having a competitor to sample DNA ratio between about 30:1 and 50:1, and other embodiments having a ratio of between about 30:1 and 40:1. In certain embodiments, 10-20 mg of yeast RNA is combined with a sample of about 200 μg-1 mg in a buffered solution with a volume of 100-300 μl. In a specific embodiment, 15 mg of yeast RNA is combined with a sample of about 400 μg in a buffered solution with a volume of 200 μl. The concentration of competitor nucleic acid in the final solution is typically between about 10 and 100 mg/ml, and may be at least about 30 mg/ml, or at least about 50 mg/ml, or at least about 70 mg/ml. In certain embodiments, the concentration of competitor nucleic acid in the final solution is about 75 mg/ml.

In samples having a high degree of complexity, increasing the hybridization time increases the opportunity for target sequences to bind to their complementary probes, even when large quantities of competitor are present. For certain embodiments of the invention, the hybridization time varies between about 10 and 100 hours, with a hybridization time between about 40 and 80 hours, or between about 55 and 65 hours in certain embodiments. The hybridization time required for a particular sample is dependent on the overall hybridization rate of the sample. In general, the hybridization rate for a single stranded sample hybridized to a complementary single stranded probe is represented by the equation t_{1/2}=ln 2/kC, where t is the hybridization time, C is the probe concentration, and k is the hybridization rate constant. The hybridization rate constant is related to the overall probe complexity—as the probe becomes more complex the hybridization rate constant decreases. See Szabo, et al., (1975) “The kinetics of in situ hybridization”, Nucleic Acid Research, 2(5): 647-53.

As indicated above, the hybridization temperature is typically chosen to be slightly lower than the melting temperature of the nucleic acid under evaluation. Conventionally, buffer solutions are chosen which provide melting temperatures of approximately 50° C. However, if the annealing time is sufficiently long (e.g., more than about 10 hours, such high temperatures can damage microarrays and other tools employed in the hybridization process). Thus, certain embodiments employ buffer conditions that permit relatively low temperature hybridization. The buffer salt can have a strong impact on melting temperature. As indicated tetraethylammonium chloride buffers may produce nucleic acid melting temperatures of approximately 30° C. Typically, the hybridization temperature will be between about 20 and 70° C. In certain embodiments, the hybridization temperature is at most about 50° C., or at most about 40° C., or at most about 30° C.

Typically, though not necessarily, the probes are immobilized on a substrate. As explained elsewhere herein, the substrate can be of many different sizes depending on the application and the type of substrate. In embodiments employing non-immobilized probes, one may attach a label such as biotin that allows the probes to be subsequently attached to a solid substrate (e.g., containing streptavidin), after hybridization. A description of annealing conditions for non-immobilized probes is found in U.S. patent application Ser. No. 11/058,432, filed Feb. 14, 2005, and titled “SELECTION PROBE AMPLIFICATION”, which is incorporated herein by reference for all purposes.

Exemplary Process

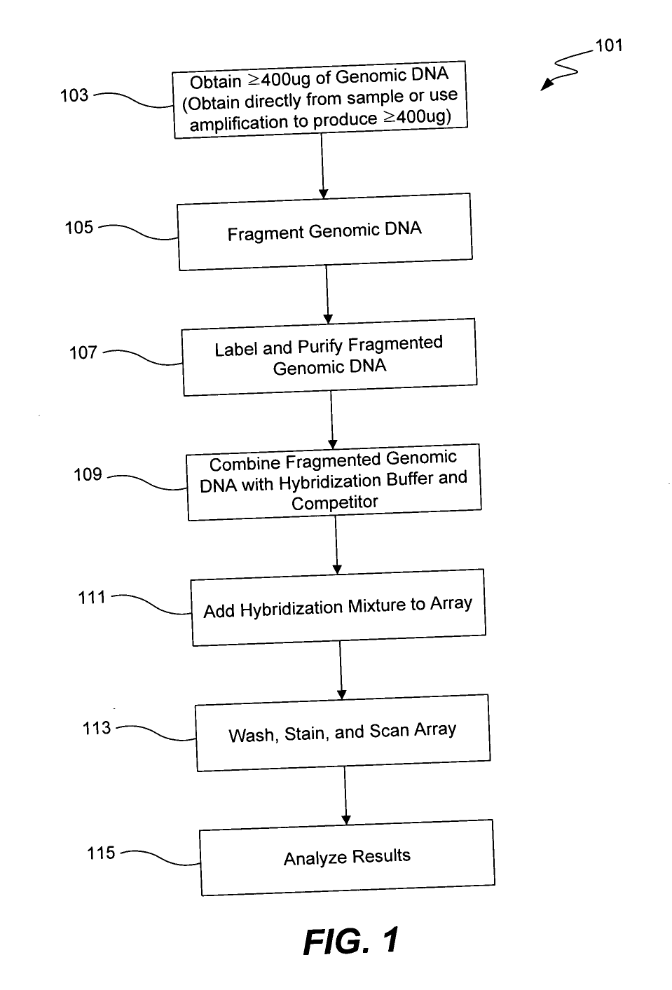

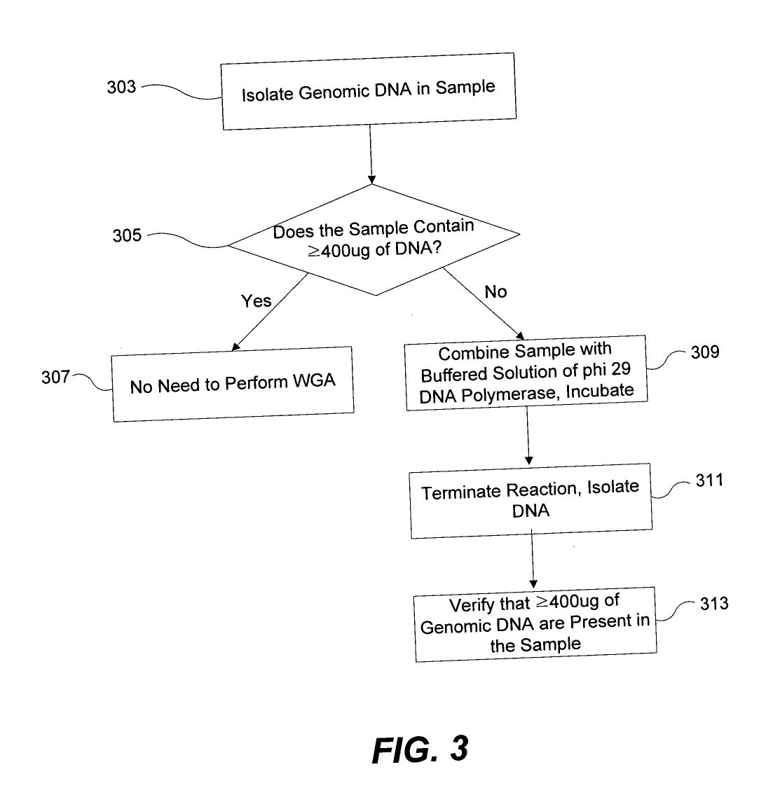

A general outline of one embodiment of this invention is given in FIG. 1. The overall method for whole genome hybridization is given by reference number 101, which begins with the aggregation of an amount of nucleic acid specified for the particular procedure, e.g., about 400 micrograms of genomic DNA. Such DNA can be obtained in its entirety from an organic sample, or an organic sample of less than 400 micrograms can be amplified by an appropriate technique such as whole genome amplification (See FIG. 3) until at least 400 micrograms of genomic DNA are obtained. See block 103. If the original sample contains a quantity of nucleic acid that is greater than the amount specified for the process, a portion of the original sample may selected and diluted as appropriate.

Once the appropriate amount of genomic DNA has been obtained, the genomic DNA sample is fragmented. See block 105. As explained below, various commonly known fragmentation techniques may be employed for this purpose. The technique chosen for a particular purpose will depend on the fragment size and end structure that is desired for a particular hybridization reaction.

Next, as depicted in block 107, the sample fragments generated in operation 105 are labeled and purified. In certain embodiments, labeling comprises the attachment of a detectable label (e.g., a fluorescent or radioactive label) to the sample fragments. In one such embodiment, labeling is performed by combining the fragmented DNA with Biotin, Terminal Deoxnucleotidyl Transferase (TdT), and a buffer. The resulting solution may be centrifuged and concentrated using a variety of well-known laboratory techniques until the labeled, fragmented DNA is concentrated into a volume of approximately 20 microliters in one embodiment.

Next, in an operation 109, the labeled, fragmented DNA from operation 107 is combined with a hybridization buffer and a hybridization competitor. In a specific embodiment, the hybridization competitor is provided in a solution comprising approximately 15 milligrams of yeast RNA. In the certain embodiments, the hybridization buffer includes tetraethylammonium chloride (TEACl), although buffers based on other hybridization reagents, such as tetramethylammonium chloride (TMACl) and/or another tetraalkylammonium halide may be used as well.

As indicated at block 111, the mixture of labeled, fragmented genomic DNA, hybridization buffer, and yeast RNA competitor is contacted with one or more probes (e.g., a hybridization array) and permitted to react with the probe(s) at a temperature appropriate for the conditions (e.g., of 30° Celsius for a TEACl-based buffer or 50° Celsius for a TMACl-based buffer). In a specific embodiment, employing a TEACl buffer, the hybridization period is about 60 hours. After hybridization is complete, the hybridization array or other source of probes employed in operation 111 is washed and stained according to common commercial techniques in step 113. Finally, in step 115, an optical or radiographic scanner scans the hybridization array of step 113 and the results are processed by, e.g., analysis software. Such software is described in detail in U.S. patent application Ser. Nos. 10/768,788, filed Jan. 30, 2004; Ser. No. 10/786,475, filed Feb. 24, 2004; or 10/970,761, filed Oct. 20, 2004. In certain embodiments, analysis software is commercially available.

Not all of the specific conditions recited for process 101 are required in all embodiments of the invention. Nor are all operations in process 101 necessary in all implementations of the invention. For example, the fragments are labeled later in the process, such as after combining with the buffer solution or even after hybridization. In other embodiments, the probes rather than the sample fragments are labeled.

In certain embodiments, the hybridization probes are not immobilized during hybridization; i.e., the sample fragments hybridize with non-immobilized single-stranded probes. In such embodiments, the probes may comprise a moiety for attachment to a solid substrate via, e.g., a biotin-streptavidin linkage. Obviously, if biotin is used for this purpose, a different type of label may be required for staining. After hybridization, the probes and associated target sequences are contacted with a solid substrate (e.g., beads, columns, plates, wafers, etc.) and permitted to become immobilized. Thereafter, the unbound sample is washed away or otherwise removed. The sequence and/or amount of hybridized target may be determined separately after separation from the immobilized probe by denaturization.

Other specific steps from the process can be generalized. Thus, an alternative characterization of the method involves the following: (1) fragmenting a nucleic acid sample to produce multiple nucleic acid fragments; (2) combining the nucleic acid fragments with a competitor in an amount that is at least about 30-fold greater than the amount of nucleic acid fragments; (3) contacting the fragments with one or more probes in the presence of the competitor under hybridization conditions that facilitate reliable detection of target sequences; and (4) selectively genotyping the nucleic acid sample only at the loci of interest (e.g. SNPs).

Two specific examples of process 101 will now be presented. In the first example, at least 400 μg of genomic DNA is obtained either directly from a biological sample or through whole genome amplification (WGA) of a biological sample. This DNA, in 180 μl of water, is fragmented by combining with 20 μl of 10× one Phor All buffer and 0.5 U of Dnase I. This mixture is incubated at 37 degrees C. for 5 minutes, then 100 degrees C. for 10 minutes. The mixture is then centrifuged to remove precipitates, and a sample of the resulting fragmented DNA is processed on a 4-20% gradient polyacrylamide gel to verify that the resulting DNA fragments are 20-300 base pairs in size, with the largest fraction of fragments in the 75-150 base pair range. The fragmented DNA is labeled by mixing with 32 μl Biotin, 4 μl 10× one Phor All buffer, and 4 μl Terminal Deoxnucleotidyl Transferase (TdT). This mixture is incubated at 37 degrees C. for 2 hours and 100 degrees C. for 10 minutes, and centrifuged to remove precipitates. The labeled DNA is purified according to one of two methods: 1) wash with 70% ethanol to precipitate the labeled DNA into a pellet and dissolve the pellet in 26 μl water, or 2) use a Centricon YM-3 column to concentrate the labeled DNA into 26 μl.

The yeast RNA competitor is prepared separately by combining 1.5 ml of a 10 mg/ml solution of yeast RNA with 0.15 ml 3M sodium acetate and 3.75 ml ethanol. This mixture is centrifuged at 11,000 rpm for 20 minutes, washed with 70% ethanol, and the resulting RNA pellet is removed and dried.

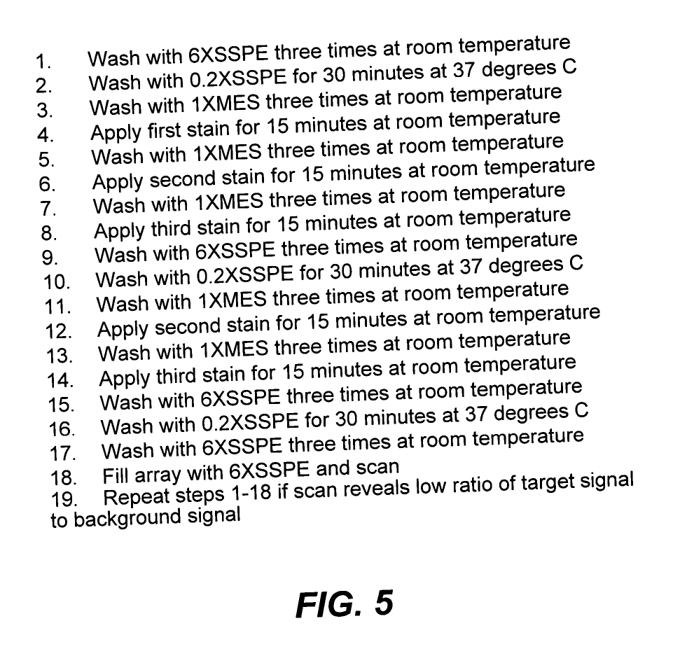

A hybridization buffer is prepared by combining 160 μl of 3M TEACl, 10 μl of 1M Tris hydrochloride, 2 μl of 1% TritonX-100, 2 μl of 5 nM control oligonucleotides and the previously purified 26 μl of labeled, fragmented DNA. (In the alternative, a TMACl buffer can be prepared by combining 120 μl of 5M TMACl, 10 μl of 1M Tris hydrochloride, 2 μl of 1% TritonX-100, 2 μl of 5 nM control oligionucleotides and the previously purified labeled, fragmented DNA in a 66 μl solution.) The buffer is added to the yeast RNA pellet previously prepared and incubated for 10 to 20 minutes at 65 degrees C. and 100 degrees C. for 10 minutes. This mixture is centrifuged to remove precipitates and injected onto a hybridization array, which is rotated at 30 to 31 degrees C. (50 degrees C. for a TMACl buffer) for 60 hours at 19 rpm. (In certain embodiments, the hybridization mixture is incubated without rotation.) The hybridization mixture is drawn off of the array and retained, while the array is washed, stained according to the procedure in FIG. 5.

In the second example, 800 μg of genomic DNA is obtained either directly from a biological sample or through whole genome amplification (WGA) of a biological sample. This DNA is dissolved in 270 μl water. 30 μl of 10× One Phor All buffer warmed to 37 degrees C. 1 μl of 0.5 U DNase I is added, the mixture is quickly mixed and incubated at 37 degrees C. for 6 minutes and 30 seconds, and 100 degrees C. for 15 minutes. The mixture is centrifuged to remove precipitates, and a sample of the resulting fragmented DNA is processed on a 4-20% gradient polyacrylamide gel to verify that the resulting DNA fragments are 20-300 base pairs in size, with the largest fraction of fragments in the 75-150 base pair range. The fragmented DNA is labeled by mixing with 64 μl Biotin, 8 μl 10× One Phor All buffer, and 8 μl Terminal Deoxnucleotidyl Transferase (TdT). This mixture is incubated at 37 degrees C. for 3 to 4 hours and 100 degrees C. for 15 minutes, and centrifuged to remove precipitates. The labeled DNA is purified by washing with 70% ethanol to precipitate the labeled DNA into a pellet and dissolving the pellet in 30 μl water.

The yeast RNA competitor is prepared separately by combining 3 ml of a 10 mg/ml solution of yeast RNA with 0.3 ml 3M sodium acetate and 7.5 ml ethanol. This mixture is centrifuged at 4 degrees C. at 11,000 rpm for 20 minutes, washed with 75% ethanol, and the resulting RNA pellet is removed and dried.

The hybridization buffer is prepared by adding to the yeast RNA pellet the 28 μl of labeled, fragmented DNA previously prepared, 160 μl of 3M TEACl, 10 μl of 1M Tris hydrochloride, 2 μl of 1% TritonX-100, and 2 μl of 5 nM control oligonucleotides. (In the alternative, a TMACl buffer can be prepared by combining 120 μl of 5M TMACl, 10 μl of 1M Tris hydrochloride, 2 μl of 1% TritonX-100, 2 μl of 5 nM control oligionucleotides and the previously purified labeled, fragmented DNA in a 66 μl solution.) This mixture is incubated at 65 degrees C. for 10 minutes and 100 degrees C. for 5 minutes. The mixture is centrifuged to remove precipitates and injected onto a hybridization array, which is rotated at 30 to 31 degrees C. (50 degrees C. for a TMACl buffer) for 60 hours at 19 rpm. (In certain embodiments, the hybridization mixture is incubated without rotation.) The hybridization mixture is drawn off of the array and retained, while the array is washed, stained according to the procedure in FIG. 5.

The Sample and its Fragments

As indicated, processes of this invention act on nucleic acid samples. In certain embodiments, the samples will have target and non-target sequences. The nucleic acid sample may be obtained from an organism under consideration and may be derived using, for example, a biopsy, a post-mortem tissue sample, and extraction from any of a number of products of the organism. In many applications of interest, the sample will comprise genomic material. The genome of interest may be that of any organism, with higher organisms such as primates often being of most interest. Genomic DNA can be obtained from virtually any tissue source. Convenient tissue samples include whole blood and blood products (except pure red blood cells), semen, saliva, tears, urine, fecal material, sweat, buccal, skin and hair. As explained, the nucleic acid sample may be DNA, RNA, or a chemical derivative thereof and it may be provided in the single or double-stranded form. RNA samples are also often subject to amplification. In this case amplification is typically preceded by reverse transcription. Amplification of all expressed mRNA can be performed, for example, as described by commonly owned WO 96/14839 and WO 97/01603.

In a specific embodiment, the target features of interest are relatively short sequences containing SNPs. As indicated above, in the case of the human genome, there are between about five million and about eight million known SNPs. This invention provides a method for efficiently isolating and amplifying sequences associated with such SNPs. Other target features (aside from SNPs) that can be isolated using the invention include insertions, deletions, inversions, translocations, other mutations, microsatellites, repeat sequences—essentially any feature that can be distinguished by its nucleic acid sequence. These features may occur, e.g., in exons or other genic regions, in promoters or other regulatory sequences, or in structural regions (e.g., centrosomes or telomeres). Regardless of whether SNPs or other features serve as targets, the invention finds use in a broad range of applications including pharmaceutical studies directed at specific gene targets (e.g., those involved in drug response or drug development), phenotype studies, association studies, studies that focus on a single chromosome or a subset of the chromosomes comprising a genome, studies that focus on expression patterns employing, e.g., probes derived from mRNA, studies that focus on coding regions or regulatory regions of the genome, and studies that focus on only genes or other loci involved in a particular disease, biochemical, or metabolic pathway. In other words, target sequences may be selected and isolated from a sample based on many different criteria or properties of interest. In other examples, target sequences are selected based on how the target sequences will be further analyzed and processed, e.g., based on the design of a DNA microarray to which the target sequences will be applied.

The amount of DNA required for whole genome hybridization is largely dependent on the size of the genome being analyzed. For the human genome, one embodiment begins with either about 400 μg of genomic DNA obtained directly from an organic sample, or a sample of less than 400 μg that has been amplified by WGA to at least 400 μg. Using WGA, a sample of genomic DNA as small as 1 ng can be amplified up to a sample of 400 μg. This amount of genomic DNA is equivalent to 10 to 30 complete copies of the human genome. Of course, larger quantities of genomic DNA can lead to more accurate results, with acceptable results obtained from quantities of human genomic DNA between 200 μg and 2 mg, with certain embodiments using between 300 μg and 800 μg of human genomic DNA (e.g., about 400 μg). The amount of sample nucleic acid in the final hybridization solution is generally between about 1 and 7 mg/ml, or between about 1.5 and 5 mg/ml.

As explained, the original nucleic acid sample may be fragmented to produce many different nucleic acid fragments, some of them harboring a target feature or sequence of interest and others not. Of course, it is possible that the initial sample will be provided in fragmented form of appropriate size and condition, which requires no separate fragmentation operation. The population of fragments may be characterized by an average size and a size distribution, as well as an occurrence rate of the target sequence. The fragmentation conditions determine these characteristics.

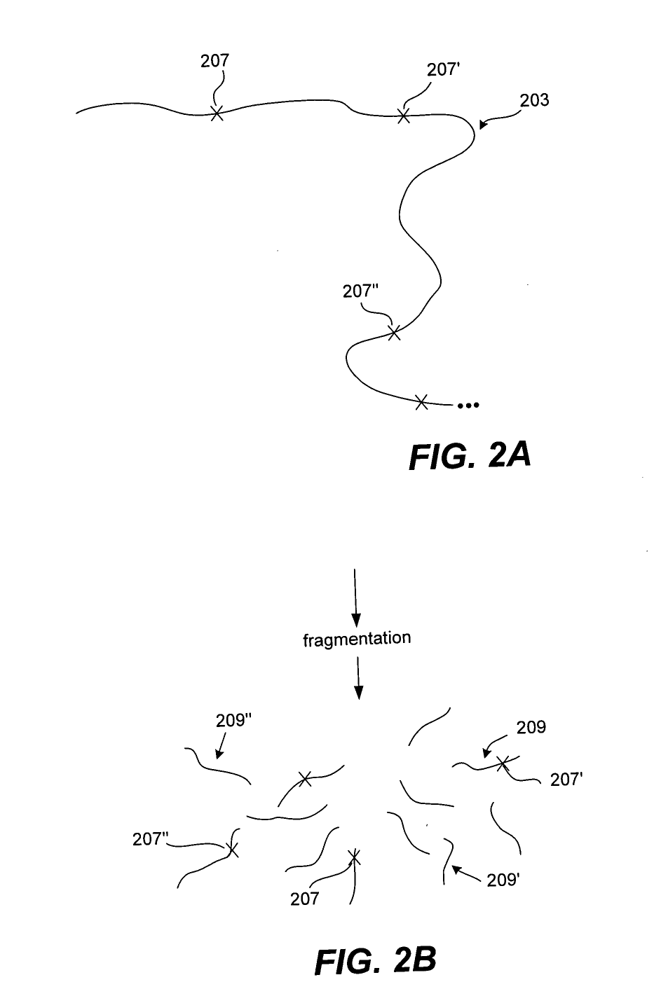

FIG. 2A depicts a continuous strand of nucleic acid 203 that may form part of a sample to be analyzed; e.g., a double-stranded segment of genomic DNA taken from a human donor. Strand 203 is shown to have multiple target features 207, 207′, 207″, . . . . These may represent SNPs or other features under investigation. At operation 103 in method 101, the sample is fragmented. This is depicted in FIG. 2B, where continuous strand 203 is fragmented into multiple strands 209, 209′, 209″, etc. Some of these strands, such as strand 209, contain a target feature of interest. Other strands such as strands 209′ and 209″ contain no target sequence.

Various considerations come into play when selecting an average or mean fragment length. In a typical case, the mean fragment size is between about 20 and 2000 base pairs in length or even longer, often between about 50 and 800 base pairs in length. In certain embodiments, the mean fragment size is between about 400 and 600 base pairs in length. In other embodiments, the mean fragment size is between about 100 and 200 base pairs in length. As one of skill will readily recognize, the optimal mean fragment length may depend on the specific application. For example, the fragment must be large enough to contain unique sequence. If hybridization will be used to select or analyze the target sequences, the fragment must be large enough to hybridize well with its complementary sequence in the particular hybridization conditions. The fragments should be small enough so that they are not easily sheared during subsequent manipulations, and so that they do not interfere with hybridization to the probes.

Another factor to consider in determining an appropriate fragment length is the final sequence analysis technique to be considered. For example, if a nucleic acid microarray is employed, the desired fragment size will be approximately 25 to 150 base pairs, or in some embodiments, between about 40 and 100.

Fragmentation of the sample nucleic acid can be accomplished through any of various known techniques. Examples include mechanical cleavage, chemical degradation, enzymatic fragmentation, and self-degradation. Self-degradation occurs at relatively high temperatures due to DNA's acidity. Methods of fragmentation may involve the use of one or more restriction enzymes. For example, one may perform a partial digestion with a mixture of restriction enzymes. Mechanical methods of fragmentation include, e.g., sonication and shearing. The fragmentation technique can provide either double-stranded or single-stranded DNA. U.S. patent application Ser. No. 10/638,113, filed Aug. 8, 2003, describes various methods, apparatus, and parameters that can be controlled to provide desired levels of fragmentation. That application is incorporated herein by reference for all purposes. In certain embodiments, enzymatic fragmentation is accomplished using a nuclease such as a DNAse. In one example, DNaseI is used. Various restriction endonucleases may be employed as well.

Amplification

While certain embodiments of the invention employ no complexity reduction such as locus-specific PCR, it is within the scope of this invention to incorporate limited complexity reduction in the process. Further as indicated above, some embodiments employ whole genome amplification.

The PCR method of amplification is generally described in PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202, each of which is incorporated by reference for all purposes. The amplification product can be RNA, DNA, or a derivative thereof, depending on the enzyme and substrates used in the amplification reaction. Certain methods of PCR amplification that may be used with the methods of the present invention are further described, e.g., in U.S. patent application Ser. No. 10,042,406, filed Jan. 9, 2002; U.S. Pat. No. 6,740,510 issued on May 25, 2004; and U.S. patent application Ser. No. 10/341,832, filed Jan. 14, 2003, each of which is incorporated herein by reference for all purposes.

Other methods exist for producing amplified sample fragments that may be employed with this invention (e.g., for isolation with probes). Some of these techniques involve other methods of tagging nucleic acid fragments, e.g., DOP-PCR, tagged PCR, etc., and are discussed in great detail in Kamberov et al. US2004/0209298 A1, which is incorporated herein by reference for all purposes.

As indicated, it may be appropriate in some cases to amplify the whole nucleic acid sample to provide a sufficient starting quantity for the hybridization process. In such cases, a process known as whole genome amplification (WGA) can be employed generate additional copies of the sample genomic DNA. There are a variety of WGA techniques available, including degenerate oligonucleotide primed PCR (DOP-PCR), tagged PCR (T-PCR), primer extension preamplification (PEP), and multiple displacement amplification (MDA). In embodiments of the invention where WGA is required, MDA is the whole genome amplification technique typically used. (For an explanation of MDA, see Dean and Hosono, “Comprehensive human genome amplification using multiple displacement amplification,” Proc Natl Acad Sci USA, 2002 Apr. 16; 99(8): 5261-5266, which is incorporated herein by reference for all purposes.)

FIG. 3 describes the optional steps involved in performing MDA-based whole genome amplification before whole genome hybridization. Genomic DNA is isolated from a sample in an operation 303. As indicated, the sample may be blood, hair, cells, or any other biological material containing genomic DNA. The sample is assayed in an operation 305 to determine if it contains a sufficient quantity of genomic DNA (labeled here as 400 ug, although the actual amount may be higher or lower). If the sample does contain a sufficient amount of genomic DNA, no WGA is required and the process can continue to FIG. 1, operation 105. If additional genomic DNA is required, the sample is mixed with a buffered solution of, e.g., phi 29 DNA polymerase. See block 309. A commercial WGA kit, such as the REPLI-g Kit from Qiagen of Valencia, Calif., can be used to perform operation 309. Next, in an operation 311, the WGA reaction is terminated and the resulting mixture of original and replicated DNA is removed. After verifying that a sufficient amount of DNA has been created by WGA in an operation 313, whole genome hybridization proceeds as shown in FIG. 1, block 105.

Probes and Probe Arrays

The probe sequence may be of any length appropriate for uniquely selecting a target sequence. In the case of target SNPs, appropriate lengths range from about 12 to 100 nucleotides, and in a more specific example they range between about 20 and 60 nucleotides in length (e.g., about 25 base pairs). Other size ranges may be appropriate for other applications.

Functionally, a “probe” is a nucleic acid capable of binding to a target nucleic acid of complementary sequence through one or more types of chemical bonds, usually through complementary base pairing, usually through hydrogen bond formation. A nucleic acid probe may include natural (i.e. A, G, C, or T) or modified bases (e.g., 7-deazaguanosine, inosine). In addition, the bases in a nucleic acid probe may be joined by a linkage other than a phosphodiester bond, so long as it does not interfere with hybridization. Thus, nucleic acid probes may be peptide nucleic acids in which the constituent bases are joined by peptide bonds rather than phosphodiester linkages.

The probes may be produced by any appropriate method including oligonucleotide synthesis techniques and isolation from organisms. In the latter case, PCR or other amplification techniques may be employed to produce the probe in relatively high concentrations. In a specific example, probes are obtained using PCR (or multiplex PCR) on sequences of the human genome found to hold specific SNPs. In such situations, the individual probes may be prepared by PCR reactions using primers specific for such probes. Such genomic sequences may be detected by any method known in the art, e.g., through association studies, linkage analysis, etc.

Many service providers make custom probes available on a contract basis. Probes for use with this invention may be ordered from such providers, some of which are the following: Agilent Technologies of Palo Alto, Calif., NimbleGen Systems, Inc. of Madison, Wis., SeqWright DNA Technology Services of Houston, Tex., and Invitrogen Corporation of Carlsbad, Calif. In another approach, the probes may be produced by fragmenting genomic DNA (e.g., a single chromosome or clone(s) from a genomic library) known to have target features. Still further, the probes may be created from mRNA by conversion to cDNA to select expressed target sequences. In other words, the expressed mRNA possesses the target sequences.

As mentioned the probes are typically, though not necessarily, immobilized. Probes may be immobilized on substrates having many different forms including bead, chips, wafers, columns, pins, optical fibers, etc. Often a plurality of probes having the same sequence are provided at a single location on a substrate or on one of many substrates (e.g., beads) and probes having a different sequence are provided at a different location of substrate.

If a DNA microarray is employed to sequence a sample, the fragments are first labeled and then contacted with the microarray under conditions that facilitate hybridization with the immobilized oligonucleotides. Any suitable label and labeling technique may be employed. Many widely used labels for this purpose provide quantifiable emission intensities, which may be detected as “signal” (e.g., target signal or background signal). In a specific example, as mentioned above, terminal transferase enzyme is employed to label the fragments. After the labels are attached to the fragments and the fragments hybridize with the oligonucleotides on the microarray, the array may be stained and/or washed to further facilitate detection of the fragments bound to the array. The binding pattern on the array is then read out and interpreted to indicate the presence or absence of the various target sequences in the sample. In the case of SNP targets, a reader identifies the alleles present in the target sequences by virtue of, for example, (1) the known sequence and location of individual probes on the array; (2) knowing that a fragment is complementary to one or more probes on the array based in its specific hybridization to the one or more probes; (3) therefore knowing the sequence of the fragment; and finally (4) therefore knowing the genotype of the fragment. Labels, oligonucleotide microarrays, and associated readers, software, etc. are provided with various conventionally available DNA microarray products such as those commercially available from, e.g., Affymetrix, Inc., (Santa Clara, Calif.). As indicated, other methods are also suitable; for example, direct sequencing of the regions encoding each marker, creation of a library comprising the target sequences, use of the target sequences as probes in further experiments or methodologies, or use in functional assays in cell lines.

Applications

This invention has many applications. In addition to genotyping individuals based on SNP alleles, the invention also permits assaying for DNA copy numbers, the presence of deletions, gene expression, loss of heterozygosity, differential allelic expression, functional genomic regions, etc. For methods related thereto, see e.g. U.S. patent application Ser. No. 09/972,595, filed Oct. 5, 2001; Ser. No. 10/142,364, filed May 8, 2002; and Ser. No. 10/845,316, filed May 12, 2004. It also introduces various efficiencies in existing genotyping methods. One of these will now be described.

As noted above, the human genome contains between five million and eight million SNPs. A single array may be able to test for ˜50,000 or more individual SNPs using current nucleic acid array technology. This is far less than the number of tests needed to perform a complete genotype. Using existing techniques, the use of multiple arrays requires the preparation of a separate DNA sample for each array, with attendant loci-specific PCR primer sets. Thus, the process of preparing multiple DNA samples for application to multiple arrays consumes significant amounts of time and money. In one embodiment of the invention, a single sample of genomic DNA is applied to more than one nucleic acid hybridization array. Because the invention is performed with little or no complexity reduction for the whole genome, it allows a single prepared sample of DNA to be serially applied to many arrays, facilitating the comparison of DNA sample to a large number of SNPs in a timely and cost effective process.

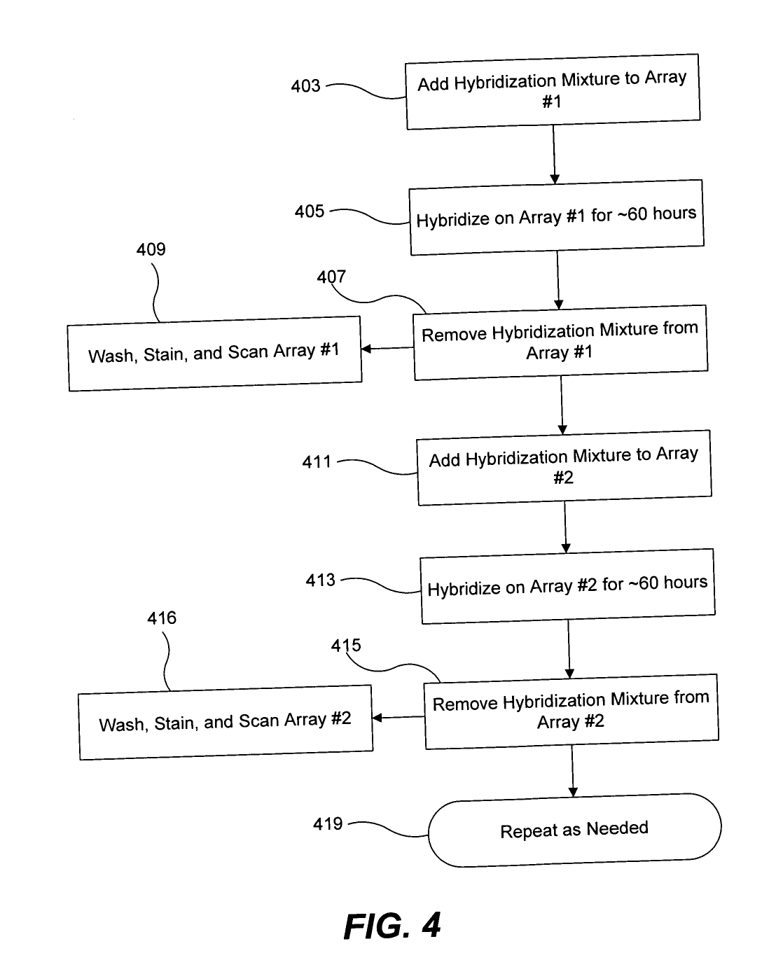

FIG. 4 depicts the sequence of operations for an embodiment of the invention in which a single sample of genomic DNA is applied to a plurality of arrays. A fragmented, labeled genomic DNA sample is combined with a hybridization buffer and added to a first array in step 403 and permitted to hybridize in step 405. Once hybridization of the DNA sample with the first array is complete, the DNA sample is removed from the first array and added to a second array in step 411. (Step 409 depicts the post-hybridization processing of the first array.) The hybridization reaction for the second array occurs in step 413, the DNA sample is removed in step 415 and the second array is processed in the same manner in step 417 as the first array was processed in step 409. Step 419 indicates that the DNA sample can be serially applied to additional arrays, as needed, until the DNA sample has been compared to the desired number of target SNPs. Using this embodiment of the invention, a single sample of genomic DNA can be compared to a number of SNPs ranging from several hundred to several million.

Generally, in nucleic acid samples, some SNPs will be easier to assay than others. This may be due to surrounding sequences, locations on particular chromosomes, sequence composition (e.g. repeat content, G-C content, complexity) etc. To address this situation, the invention may be employed to identify a collection of “working SNPs” selected to genotype individual humans (or among some other amount employed, as necessary, to genotype individuals of other species). The working SNPs are selected based upon their ability to reproducibly and reliably hybridize with the probes in the presence of competitor and under hybridization conditions of this invention. As such, the present invention may be employed to identify those SNPs or other genetic features that perform better than their peers in assays using the invention.

This aspect of the invention may be understood as a method of identifying a set of working single nucleotide polymorphisms (SNPs) from among a larger group of SNPs in a genome. One outline of the process includes the following operations: (a) providing a genomic nucleic acid sample of at least about 400 MB complexity having a plurality of sequences comprising SNPs, where some of said sequences reliably hybridize with a specified collection of hybridization probes (e.g., a microarray) and others do not; (b) providing fragments of the genomic nucleic acid sample in a buffer solution having a competitor nucleic acid in an amount of between about 30-fold and 40-fold greater than an amount of the genomic nucleic acid sample in the buffer solution; (c) contacting the fragments of the genomic nucleic acid sample in the buffer solution with multiple hybridization probes complementary to at least some of the plurality of sequences comprising SNPs; (d) determining which of the sequences comprising SNPs reliably hybridize with said multiple hybridization probes in (c); and (e) selecting a set of working SNPs based on at least some of the sequences comprising SNPs that reliably hybridize. While this example describes SNPs, it could just as well apply to other genetic features such as insertions, deletions, etc.

SNPs that reliably hybridize are, in certain embodiments, those SNPs for which the analysis of hybridization results in the identification or “calling” of a genotype for the SNP. In other words, the genotypes for SNPs that reliably hybridize can be “called” and those for SNPs that do not reliably hybridize cannot be “called.” A method for determining whether a SNP genotype can be called is dependent on the hybridization assay being used. In certain embodiments, such a method is a multistep process dependent on a plurality of criteria, e.g., extend of target signal, extent of background signal, the ratio of target signal to background signal, concordance of the results with other genotyping methods, statistical analyses (e.g., likelihood calculations, Hardy-Weinberg equilibrium analysis), etc. Specific examples of methods for determining genotypes using such metrics derived from DNA microarray hybridization analyses are detailed in, e.g., U.S. patent application Ser. Nos. 10/768,788, filed Jan. 30, 2004; Ser. No. 10/786,475, filed Feb. 24, 2004; or 10/970,761, filed Oct. 20, 2004.

Some applications of the invention may be implemented using kits or other combinations containing a hybridization competitor, a buffer salt, and one or more probes complementary to one or more target sequences within a nucleic acid sample. In certain embodiments, the buffer salt comprises a tetraalkylammonium halide such as tetraethylammonium chloride. The kit optionally comes with instructions for using the elements of the kit to conduct, e.g., a hybridization assay. The instructions can explain one or more of the following: preparation of the nucleic acid sample, hybridization conditions, how to add competitor, and how to prepare a buffer solution. In certain embodiments, the instructions explain how to prepare a buffer in which the competitor nucleic acid is present at a concentration of between about 10 mg/ml and about 100 mg/ml, or a buffer in which the competitor nucleic acid is present at a concentration of at least about 30 mg/ml, or at least about 50 mg/ml, or at least about 75 mg/ml. The content of the instructions may follow the methodologies set forth above.

For kits, the hybridization competitor is generally an RNA or other moderately to highly soluble nucleic acid. In certain embodiments, the kit also includes an enzyme or other reagent for fragmenting the genomic nucleic acid sample. As indicated, one such enzyme is a DNAse. The kit may also include primers and polymerase for amplifying the whole nucleic acid sample. The probes may be provided as one or more nucleic acid microarrays, beads, columns or the like containing nucleic acid oligomers for detecting target sequences contained within the target fragments.

Additionally, the kits may comprise a label for labeling fragments of the genomic nucleic acid sample. The label can bind with a stain or other signal-producing component employed after hybridization has occurred. In a specific embodiment, the label is biotin and the stain or other signal-producing component comprises an avidin moiety. The kits may further comprise a stain (e.g., a fluorophore), radio-label, quantum dot, or the like for producing a signal to indicate which probes have hybridized with labeled fragments.

### Other Embodiments

The present invention has a broader range of implementation and applicability than described above. Therefore, it is to be understood that the above description is intended to be illustrative and not restrictive. It should be readily apparent to one skilled in the art that various embodiments and modifications may be made to the invention disclosed in this application without departing from the scope and spirit of the invention. The scope of the invention should, therefore, be determined not with reference to the above description, but should instead be determined with reference to the appended claims, along with the full scope of equivalents to which such claims are entitled. All publications mentioned herein are cited for the purpose of describing and disclosing reagents, methodologies and concepts that may be used in connection with the present invention. Nothing herein is to be construed as an admission that these references are prior art in relation to the inventions described herein. Throughout the disclosure various patents, patent applications and publications are referenced. Unless otherwise indicated, each is incorporated by reference in its entirety for all purposes.

What is claimed is:

1. A method of hybridizing a genomic nucleic acid sample to one or more probes complementary to one or more target sequences within the genomic nucleic acid sample, the method comprising:
(a) providing the genomic nucleic acid sample, wherein the genomic nucleic acid sample comprises a sequence with a complexity of at least about 400 MB representing at least a portion of the genome of an organism;
(b) contacting the genomic nucleic acid sample with a buffer solution comprising a competitor nucleic acid in an amount of at least about 30-fold greater than an amount of the genomic nucleic acid sample; and
(c) allowing the genomic nucleic acid sample in the buffer solution to contact said one or more probes and permit hybridization,
wherein the genomic nucleic acid sample does not undergo complexity reduction before hybridization with said one or more probes.

2. The method of claim 1, wherein the one or more probes are immobilized on at least one substrate.

3. The method of claim 1, wherein the one or more probes comprise multiple probes of distinct sequences immobilized on one or more substrates.

4. The method of claim 1, wherein the one or more probes are provided on one or more microarrays of nucleic acid probes.

5. The method of claim 1, wherein the one or more probes comprise oligonucleotides of between about 12 and 100 nucleotides in length.

6. The method of claim 1, wherein the one or more probes comprise oligonucleotides of between about 20 and 60 nucleotides in length.

7. The method of claim 1, wherein the genomic nucleic acid sample has a complexity of at least about 1 GB.

8. The method of claim 1, wherein the genomic nucleic acid sample has a complexity of at least about 3 GB.

9. The method of claim 1, wherein the genomic nucleic acid sample comprises a whole genome of an organism.

10. The method of claim 1, wherein the genomic nucleic acid sample comprises at least a portion of a human genome.

11. The method of claim 1, wherein the competitor nucleic acid comprises RNA.

12. The method of claim 1, wherein the competitor nucleic acid is present in the buffer solution at a concentration of at least about 10 mg/ml.

13. The method of claim 1, wherein the buffer solution further comprises a salt.

14. The method of claim 13, wherein the salt comprises a tetraalkylammonium salt.

15. The method of claim 14, wherein the tetraalkylammonium salt is TEACl (tetraethylammonium chloride).

16. The method of claim 15, wherein the buffer solution is maintained at a temperature of at most about 40° C. during contact with said one or more probes.

17. The method of claim 14, wherein the tetraalkylammonium salt is TMACl (tetramethylammonium chloride).

18. The method of claim 17, wherein the buffer solution is maintained at a temperature of at most about 70° C. during contact with said one or more probes.

19. The method of claim 1, wherein the buffer solution contacts said one or more probes for a duration of between about 10 and 100 hours.

20. The method of claim 1, wherein the genomic nucleic acid sample does not undergo complexity reduction by size fractionation, restriction enzyme digestion, locus-specific PCR, and/or subtractive hybridization.

21. The method of claim 1, wherein the competitor nucleic acid has a solubility of at least about 50 mg/ml of the buffer solution.

22. The method of claim 1, wherein the competitor nucleic acid is present in the buffer solution in an amount of between about 30-fold and 40-fold greater than an amount of the genomic nucleic acid sample.

23. A method of preparing a complex genomic sample for analysis, the method comprising:
(a) providing a genomic nucleic acid sample having a complexity of at least about 4×10⁸ base pairs;
(b) fragmenting the genomic nucleic acid sample to produce multiple fragments of the sample;
(c) incorporating the fragments into a buffer solution comprising:
(i) a competitor nucleic acid serving as a hybridization competitor, and
(ii) a salt which causes the fragments of the genomic nucleic acid to have a melting temperature of between about 20 and 70° C.; and
(d) contacting the buffer solution with one or more hybridization probes and allowing said one or more hybridization probes to hybridize with the fragments of the genomic nucleic acid sample.

24. The method of claim 23, further comprising staining at least some fragments of the genomic nucleic acid samples which hybridized with said one or more hybridization probes to thereby facilitate analysis.

25. The method of claim 23, wherein the genomic nucleic acid sample has a complexity of at least about 1×10⁹ base pairs.

26. The method of claim 23, wherein the genomic nucleic acid sample comprises a substantially whole genome of an organism.

27. The method of claim 26, further comprising performing whole genome amplification on the genomic nucleic acid sample.

28. The method of claim 23, wherein fragmenting the genomic nucleic acid sample comprises contacting said sample with a DNAse.

29. The method of claim 23, wherein the fragments of the sample have an average length of between about 50 and 500 base pairs.

30. The method of claim 23, wherein the salt comprises a tetraalkylammonium salt.

31. The method of claim 30, wherein the tetraalkylammonium salt is tetraethylammonium chloride.

32. The method of claim 31, wherein said contacting the buffer solution with one or more hybridization probes is carried out at a temperature of between about 20° C. and 40° C.

33. The method of claim 30, wherein the tetraalkylammonium salt is tetramethylammonium chloride.

34. The method of claim 33, wherein said contacting the buffer solution with one or more hybridization probes is carried out at a temperature of between about 50° C. and 70° C.

35. The method of claim 23, wherein the competitor nucleic acid comprises RNA.

36. The method of claim 35, wherein the RNA is present in the buffer solution in an amount of between about 10 mg/ml and about 100 mg/ml.

37. The method of claim 23, wherein the competitor nucleic acid has a solubility of at least about 50 mg/ml of the buffer solution.

38. The method of claim 23, wherein said contacting the buffer solution with the one or more hybridization probes takes place for a period of between about 10 and 100 hours.

39. The method of claim 23, wherein said contacting the buffer solution with the one or more hybridization probes takes place for a period of between about 20 and 70 hours.

40. A kit for analyzing a complex genomic nucleic acid sample, the kit comprising:
(a) a hybridization competitor comprising a competitor nucleic acid;
(b) a buffer salt comprising tetraethylammonium chloride; and
(c) one or more probes complementary to one or more target sequences within the genomic nucleic acid sample.

41. The kit of claim 40, further comprising an enzyme for fragmenting the genomic nucleic acid sample.

42. The kit of claim 41, wherein the enzyme comprises a DNAse.

43. The kit of claim 40, further comprising a label for fragments of the genomic nucleic acid sample.

44. The kit of claim 43, wherein said label comprises biotin.

45. The kit of claim 40, further comprising a stain for fragments of the genomic nucleic acid sample that hybridize with the one or more probes.

46. The kit of claim 40, wherein the one or more probes are provided on a nucleic acid microarray.

47. The kit of claim 40, wherein said kit is employed to analyze a genomic nucleic acid sample having a complexity of at least about 400 MB.

48. The kit of claim 40, further comprising instructions for preparing a buffer in which the competitor nucleic acid is present at a concentration of between about 10 mg/ml and about 100 mg/ml.

49. The kit of claim 40, wherein the competitor nucleic acid has a solubility of at least about 50 mg/ml of buffer solution.

50. The kit of claim 40, wherein the competitor nucleic acid is RNA.

51. A method of identifying a set of working single nucleotide polymorphisms (SNPs) from among a larger group of SNPs in a genome, the method comprising:
(a) providing a genomic nucleic acid sample of at least about 400 MB complexity having a plurality of sequences comprising SNPs, wherein some of said sequences reliably hybridize with a specified collection of hybridization probes and others do not reliably hybridize with said hybridization probes;
(b) providing fragments of said genomic nucleic acid sample in a buffer solution having a competitor nucleic acid in an amount of between about 30-fold and 40-fold greater than an amount of the genomic nucleic acid sample in the buffer solution;
(c) contacting the fragments of said genomic nucleic acid sample in the buffer solution with multiple hybridization probes complementary to at least some of the plurality of sequences comprising SNPs;
(d) determining which of said sequences comprising SNPs reliably hybridize with said multiple hybridization probes in (c); and
(e) selecting SNPs from at least some of the sequences comprising SNPs that reliably hybridize as a set of working SNPs.

52. A hybridization solution comprising:
(a) a fluid medium;
(b) a fragmented genomic nucleic acid sample of at least about 400 MB complexity in the liquid medium;
(c) a hybridization competitor nucleic acid present in an amount of between about 30-fold and 40-fold greater than an amount of the genomic nucleic acid sample in the fluid medium; and
(d) a buffer salt comprising tetraethylammonium chloride in the fluid medium.
